(19) United States
(12) Patent Application Publication
(10) Pub. No.: US 2023/0375514 A1
(43) Pub. Date: Nov. 23, 2023

(54) THIOCARBAMATE-BASED INDICATOR DETECTION OF OZONE

(71) Applicant: Hach Company, Loveland, CO (US)

(72) Inventors: Angella Nicholle Greenawalt, Fort Collins, CO (US); Amit Das, Fort Collins, CO (US)

(21) Appl. No.: 18/319,143

(22) Filed: May 17, 2023

Related U.S. Application Data
(60) Provisional application No. 63/343,808, filed on May 19, 2022.

(51) Int. Cl. G01N 31/22; G01N 33/18

(52) U.S. Cl. CPC G01N 31/225; G01N 33/18

(57) ABSTRACT

An embodiment provides a method for measuring ozone in a sample, including: preparing a thiocarbamate-based indicator; introducing the thiocarbamate-based indicator to a sample, wherein the sample contains an amount of ozone and the introducing causes a change in fluorescence of the solution; and measuring the amount of ozone in the sample by measuring the change in intensity of the fluorescence. Other aspects are described and claimed.

### CROSS REFERENCE TO RELATED APPLICATION

This application claims priority to U.S. Provisional Patent Application Ser. No. 63/343,808, filed on May 19, 2022, and entitled “THIOCARBAMATE-BASED INDICATOR DETECTION OF OZONE,” the contents of which are incorporated by reference herein.

### BACKGROUND

This application relates generally to measuring ozone in aqueous or liquid samples, and, more particularly, to the measurement of ozone using a thiocarbamate-based indicator.

Ensuring water quality is critical in a number of industries such as pharmaceuticals and other manufacturing fields. Additionally, ensuring water quality is critical to the health and well-being of humans, animals, and plants which are reliant on the water for survival. One element that is typically measured is ozone. Ozone may be controlled to make water safe for humans, animals, and aquatic life. Therefore, detecting the presence and concentration of ozone in water, food materials, or other liquid solutions is vital.

### BRIEF SUMMARY

In summary, one embodiment provides a method for measuring ozone in a sample, comprising: preparing a thiocarbamate-based indicator; introducing the thiocarbamate-based indicator to a sample, wherein the sample contains an amount of ozone and the introducing causes a change in fluorescence of the solution; and measuring the amount of ozone in the sample by measuring the change in intensity of the fluorescence.

Another embodiment provides a method for measuring ozone in a sample, comprising: preparing a thiocarbamate-based indicator; introducing the thiocarbamate-based indicator to a sample, wherein the sample contains an amount of ozone and the introducing causes a change in fluorescence of the solution; adding potassium iodide to the sample, wherein the potassium iodide accelerates the reaction rate between the thiocarbamate-based indicator and ozone and causes a change in fluorescence of the sample; and measuring the amount of ozone in the sample by measuring the change in intensity of the fluorescence.

A further embodiment provides a measurement device which measures ozone in a sample, comprising: a thiocarbamate-based indicator; at least one measurement chamber; a processor; and a memory storing instructions executable by the processor to: introduce the thiocarbamate-based indicator to a sample, wherein the sample contains an amount of ozone; add potassium iodide to the sample, wherein the potassium iodide accelerates the reaction rate between the thiocarbamate-based indicator and ozone and causes a change in fluorescence of the sample; and measure the amount of ozone in the sample by measuring the change in fluorescence of the sample.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a flow diagram of an example ozone measuring system for a sample.

FIG. 2 illustrates a reaction scheme example of a thiocarbamate-based indicator for detection of ozone.

FIG. 3 illustrates an example ozone calibration curve using a thiocarbamate-based indicator from two operators.

FIG. 4A illustrates example data of ozone measurement using a thiocarbamate-based indicator.

FIG. 4B illustrates further example data of ozone measurement using a UV-Vis detection method.

FIG. 5 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Conventional methods of ozone measurement in water may have some limitations. For example, ozone measurement may be used to determine the quality of water. High concentrations of ozone may be harmful to animals, humans, and/or plants. Accordingly, as another example, a user or entity may want the ozone in a body of water to be under a particular threshold, therefore, the user may measure the ozone to determine if the amount of ozone is under that threshold.

A standard for free and ozone measurement in water is Hach's AccuVac available from Hach Company, Loveland CO, USA (AccuVac is a registered trademark of Hach Company in the United States and other countries) which is a bleaching chemistry. These methods result in a bleaching of color in an amount proportional to the ozone concentration. The resulting color from the colorimetric reaction may be determined photometrically, for example, using a spectrophotometer. The amount of ozone may be determined by comparison to a similarly prepared blank vial. The absorbance of the sample reacted vial must be compared to the absorbance of the unreacted blank vial to determine the ozone concentration of the sample reacted vial.

However, the current analyte testing methods have limitations which are overcome by the methods and techniques as described in more detail herein. One limitation of the current techniques is that they use a bleaching chemistry not favorable to some users and measurement systems. Additionally, the traditional colorimetric methods require the preparation of a separate “blank” vial. The extra step of preparing a blank vial can introduce error to the measurement based upon individual human techniques in preparing the blank. Also, since the traditional colorimetric technique involves the bleaching of a dye, the time for preparation and time a measurement is taken, can introduce variability in the sample reading. Additionally, because the techniques include bleaching of a dye, difficulty may arise because there may not be the same volume of starting colorimetric dye in both the blank and sample vial, thereby introducing error into the determination of the amount of analyte found in the sample. This error may result in a false positive or false negative result.

Accordingly, an embodiment provides a system and method for measuring ozone in a sample. The sample may be drawn from a volume of liquid such as a holding tank, water source, food material source, beverage source, or the like. The sample may be free of other oxidants. In an embodiment, the method may detect ozone in concentrations in about the range of 0 to 1.5 parts per million (ppm). In an embodiment, the method may use a fluorometric method. The indicator to give a fluorescent signal may be a thiocarbamate derivative. The thiocarbamate derivative may be a derivative of 7-hydroxy coumarin. The thiocarbamate may be an umbelliferone thiocarbamate. An additive may be added to the method. The additive may accelerate the completion time of a reaction. The additive may be potassium iodide (KI). In an embodiment, the fluorescence may be correlated to the detection of ozone. In an embodiment, the fluorescence intensity or change in fluorescence may be correlated to ozone in a sample. A buffer such as phosphate buffer may be added. In an embodiment, the pH of a solution may be adjusted to activate the reporter or indicator molecule. The pH may be adjusted to about pH 4.0 to about 8.5.

The illustrated example embodiments will be best understood by reference to the figures. The following description is intended only by way of example, and simply illustrates certain example embodiments.

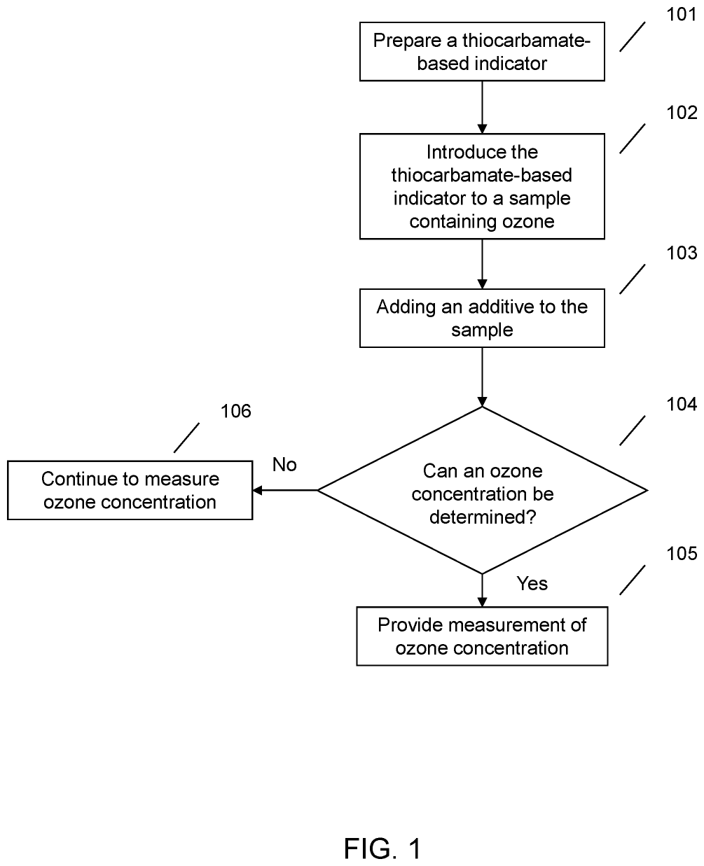

Referring to FIG. 1, an example system and method for detection of ozone in a sample is illustrated. In an embodiment, a thiocarbamate-based indicator may be prepared. The thiocarbamate-based indicator may be introduced to a sample containing ozone. In an embodiment, an additive may be added. The additive may be potassium iodide (KI). The additive may accelerate a reaction. In another embodiment, the additive or KI may not be added. In an embodiment, the thiocarbamate-based indicator in the presence of ozone may cause a change in fluorescence intensity of the thiocarbamate-based indicator. The change of fluorescent intensity may be correlated to a concentration of ozone in the solution or sample.

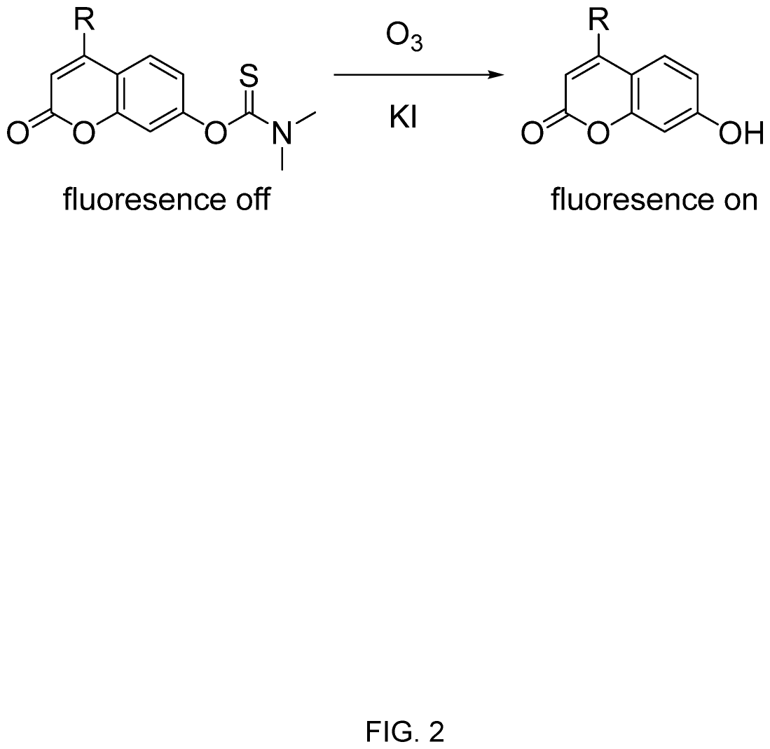

At 101, in an embodiment, a thiocarbamate-based indicator may be prepared. The thiocarbamate-based indicator may be a thiocarbamate derivative of hydroxy coumarin. In an embodiment, the thiocarbamate-based indicator may be methylumbelliferone thiocarbamate or umbelliferone thiocarbamate. Referring to FIG. 2, an example reaction of the thiocarbamate-based indicator is illustrated. In an embodiment, the thiocarbamate-based indicator may detect ozone in the range of 0-1.5 mg/L. The range is exemplary, a range may be determined based upon the need to control ozone to a suitable level for water treatment for example.

At 102, in an embodiment, the thiocarbamate-based indicator may be introduced into a sample. The sample may contain ozone or an amount of ozone. The solution may be a sample which may include a sample from a natural body of water, a holding tank, a processing tank, a pipe, a water system, a volume of liquid for food preparation, or the like. The solution may be in a continuous flow, a standing volume of liquid, or any combination thereof. In one embodiment, the sample may be introduced to the thiocarbamate-based indicator, for example, a test chamber of the measurement device. Introduction of the sample into the measurement device may include placing or introducing the sample into a test chamber manually by a user or using a mechanical means, for example, gravity flow, a pump, pressure, fluid flow, or the like. For example, a water sample for ozone testing may be introduced to a measurement or test chamber using a pump. In an embodiment, valves or the like may control the influx and efflux of the solution into or out of the one or more chambers, if present.

Additionally, or alternatively, the measurement device may be present or introduced in a volume of the sample. The measurement device is then exposed to the volume of sample where it can perform measurements. The system may be a flow-through system in which a solution and/or reagents are automatically mixed and measured. Once the sample is in contact with the measurement system, the system may measure the ozone of the sample or a change in fluorescence of the sample, as discussed in further detail herein. In an embodiment, the measurement device may include one or more chambers in which the one or more method steps may be performed.

At 103, in an embodiment, an additive may be added to the sample. The additive may be an optional step. The additive may be potassium iodide (KI). The additive may accelerate the reaction. The additive may accelerate the reaction of the thiocarbamate-based indicator and the ozone. The additive may reduce the reaction time and/or accelerate the reaction of the thiocarbamate-based indicator with ozone to approximately 30 seconds. In an embodiment, the pH of the solution may be controlled. Additionally, or alternatively, ozone may be added to the solution. The ozone may be added to a sample using an ozone generator or may be present from another process. In an embodiment, the thiocarbamate-based indicator in the presence of ozone may “turn-on” the fluorescent properties of the thiocarbamate-based indicator.

In an embodiment, the pH of the solution may be adjusted. For example, the pH may be adjusted or titrated to around a pH in the range of about 4.0 to about 8.5. The thiocarbamate-based indicator concentration may be approximately 5-40 micromolar (μM). The indicator concentration may be adjusted based upon an ozone concentration in a sample. In an embodiment, a buffer may be added. The buffer may be a phosphate buffer. The phosphate buffer concentration may be about 75 mM. In an embodiment, the potassium iodide may be approximately 30-40 μM. An approximate range of detection of ozone is between 0-1.5 mg/L.

In an embodiment, a co-solvent may be added to the sample. The co-solvent may allow the thiocarbamate-based indicator to be more soluble in. The co-solvent may be a low molecular weight molecule. In an embodiment, the co-solvent may comprise isopropanol, ethanol, poly(ethylene glycol), poly(ethylene glycol)dimethyl ether, poly(ethylene glycol)methyl ether, or the like. In an embodiment, the co-solvent may be a mixture of the co-solvents contemplated. The co-solvent may be used at a concentration less than or equal to 10% of the sample volume.

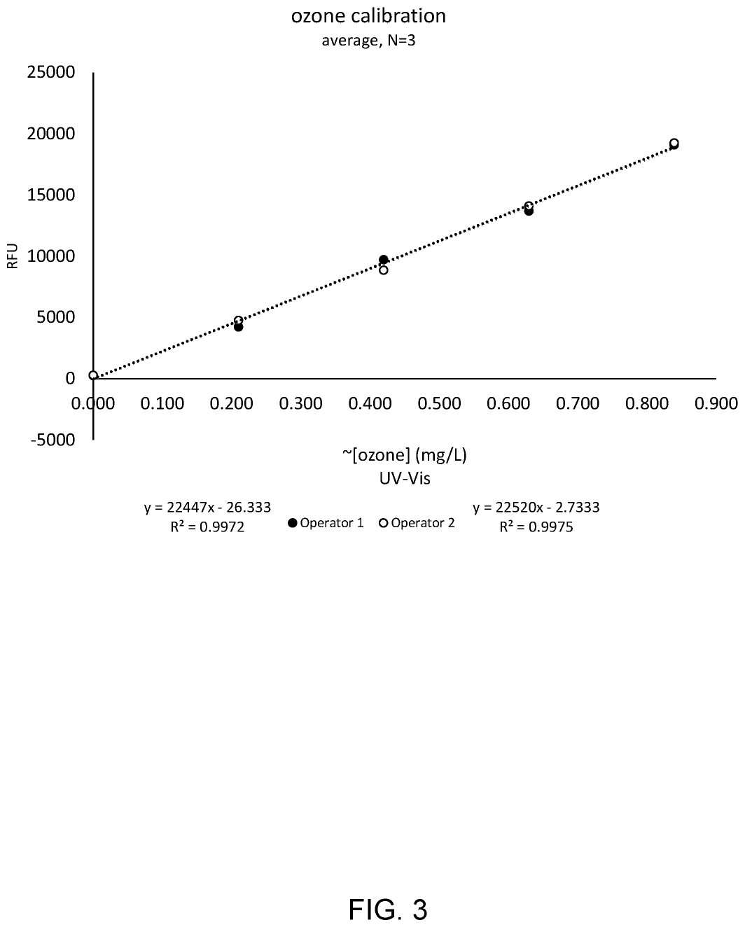

At 104, in an embodiment, the system and method may determine if an ozone concentration may be determined. In an embodiment, the presence of ozone in a sample may cause an increase in fluorescence intensity of the thiocarbamate-based indicator. Examples of this increase in fluorescence intensity and dose response curves for a thiocarbamate-based indicator may be illustrated in FIG. 3. An embodiment, of relative fluorescence units (RFU) is plotted over ozone concentration (milligrams/Liter). Data from two operators is illustrated. An operator may refer to lab personnel or technician. The illustrated ozone concentration is from 0 to 0.839 mg/L. Experimental conditions may include generation of ozone with an ozone generator, at room temperature or chilling the sample to around 0-5 degrees Celsius, stirring the sample, pretreating the pipette tip by placing in the ozonated sample, and using a 10 cm cell to measure absorbance. These conditions are exemplary and may be altered based upon conditions. In an embodiment, the thiocarbamate-based indicator may be used at a final concentration 5-40 micromolar (μM).

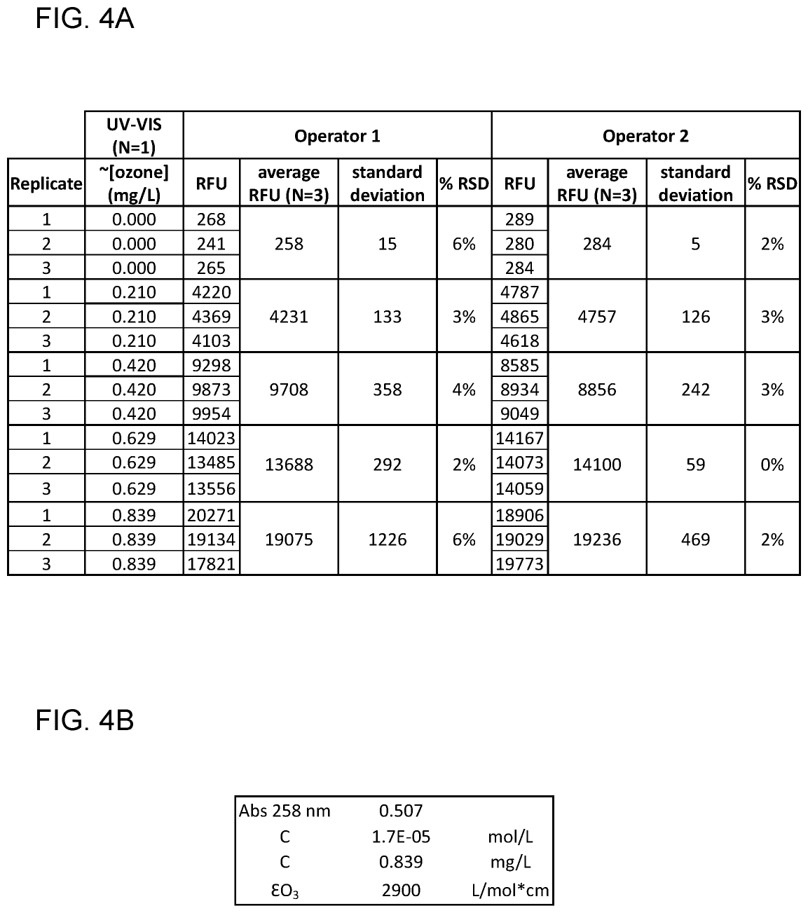

The measured absorbance may be correlated to a concentration or amount of ozone in the sample or water. The correlation may be based off a UV-Vis measurement for ozone. This may be used as a reference measurement. The same aliquot may then be used in a 16 cm cell with buffer and an indicator. The sample may be diluted with deionized water depending on the ozone concentration. Measurements may be made using the Hach DR1300 FL (available from Hach Company, Loveland CO, USA) instrument to measure the RFU value or concentration versus the UV-Vis value. Operator refers to lab personnel performing the experiment. Referring to FIG. 4A, in an embodiment, example data are illustrated using a thiocarbamate-based indicator to measure ozone concentration. Replicates by different operators are shown for different ozone concentration demonstrating the performance and accuracy of the thiocarbamate-based indicator as an ozone measurement method. In an embodiment, an excitation wavelength may be between 320 and 385 nanometers (nm). In an embodiment, an emission wavelength may be 420-470 nm. Referring to FIG. 4B, in an embodiment, Beer's Law is used to calculate the concentration of ozone using a 10 cm pathlength and a known molar extinction coefficient at a wavelength of 258 nm.

A phosphate buffer may be added. A solution of the thiocarbamate-based indicator may be prepared in isopropanol and added to the sample keeping the isopropanol volume of less than or equal to 10% of the sample volume. In an embodiment citrate may be added as an additive. The concentration of the citrate may be 15 mM. The citrate may prevent the formation of a metal phosphate by complexation. Potassium iodide may be added as a catalytic additive.

Therefore, the fluorescence intensity, of a solution containing ozone may be correlated to the intensity of a change in the intensity in the sample or aqueous solution. Fluorescence curves may be generated for a range of concentrations, for different thiocarbamate-based indicators, for any different condition that may affect absorption or fluorescence values (e.g., temperature, sample content, turbidity, viscosity, measurement apparatus, aqueous sample chamber, etc.), or the like.

Alternatively, or additionally, ozone measurement may be at periodic intervals set by the user or preprogrammed frequencies in the device. Measurement by a device allows for real time data with very little human involvement in the measurement process. Cleaning of the fluorometric chamber may be required at an unspecified time interval. A programmed calibration curve may be entered into the device.

A chamber, vessel, cell, chamber, or the like may contain a sample, at least one thiocarbamate-based indicator, and associated reagents such as buffers and/or additives. A device may contain one or more bottles of reagents which contain necessary reagents. The reagents contained in the one or more bottles may be pump fed or gravity fed. The flow of the reagents may be metered to ensure proper volume delivery to the measurement cell. The sample may be fed through a pressured inlet, a vessel, or the like. The sample may be introduced into the measurement chamber by a pump or gravity fed. The sampling device may be in series or parallel to an aqueous flow. The device may have a system to ensure proper mixing of the aqueous sample, thiocarbamate-based indicator, and related reagents.

The fluorescent intensity or ozone concentration may be an output upon a device in the form of a display, printing, storage, audio, haptic feedback, or the like. Alternatively, or additionally, the output may be sent to another device through wired, wireless, fiber optic, Bluetooth®, near field communication, or the like. An embodiment may use an alarm to warn of a measurement or concentration outside acceptable levels. An embodiment may use a system to shut down water output or shunt water from sources with unacceptable levels of an analyte. For example, an analyte measuring device may use a relay coupled to an electrically actuated valve, or the like.

At 106, in an embodiment, if a concentration of ozone cannot be determined, the system may continue to measure ozone, changes in fluorescence intensity and/or fluorescence intensity. Additionally, or alternatively, the system may output an alarm, log an event, or the like.

If a concentration of ozone can be determined, the system may provide a measurement of ozone concentration at 105. The system may connect to a communication network. The system may alert a user or a network. This alert may occur whether an ozone measurement is determined or not. An alert may be in a form of audio, visual, data, storing the data to a memory device, sending the output through a connected or wireless system, printing the output or the like. The system may log information such as the measurement location, a corrective action, geographical location, time, date, number of measurement cycles, or the like. The alert or log may be automated, meaning the system may automatically output whether a correction was required or not. The system may also have associated alarms, limits, or predetermined thresholds. For example, if an ozone concentration reaches a threshold. Alarms or logs may be analyzed in real-time, stored for later use, or any combination thereof.

The various embodiments described herein thus represent a technical improvement to conventional ozone measurement techniques. Using the techniques as described herein, an embodiment may use a thiocarbamate-based indicator to measure ozone in solution. This is in contrast to methodology with limitations mentioned above. Such techniques provide a faster and more accurate method for measuring ozone in an aqueous or liquid solution. The various embodiments described herein thus represent a technical improvement to precise ozone measurement in a sample. Using the techniques as described herein, an embodiment may use a method and device to measure ozone concentration. This is in contrast to conventional methods with limitations mentioned above.

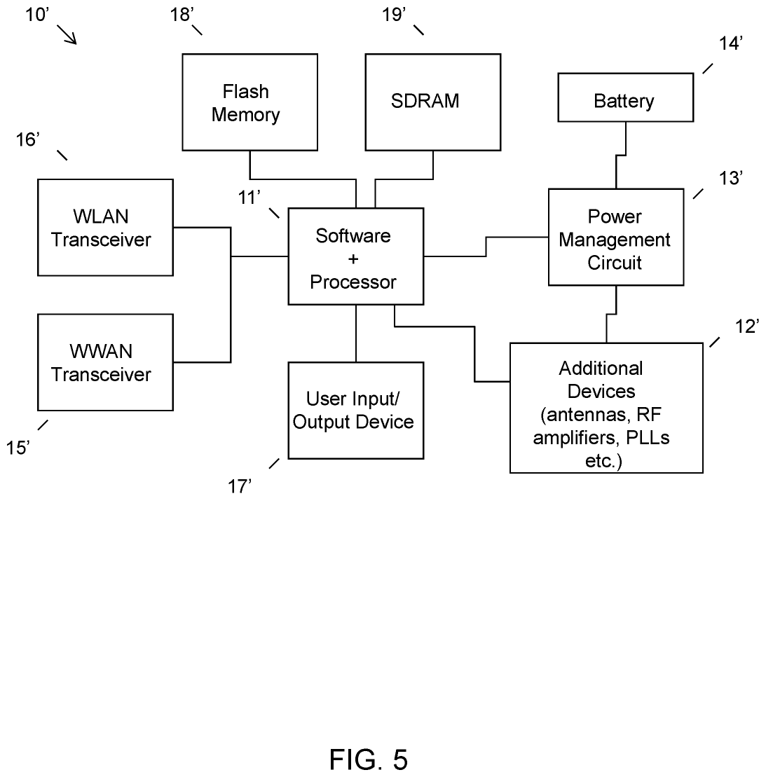

While various other circuits, circuitry or components may be utilized in information handling devices, with regard to an instrument for ozone measurement in according to any one of the various embodiments described herein, an example is illustrated in FIG. 5. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment to perform ozone measurement of a sample.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a handheld measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for measuring ozone in a sample, comprising:
preparing a thiocarbamate-based indicator;
introducing the thiocarbamate-based indicator to a sample, wherein the sample contains an amount of ozone and the introducing causes a change in fluorescence of the solution; and
measuring the amount of ozone in the sample by measuring the change in intensity of the fluorescence.

2. The method of claim 1, wherein the thiocarbamate-based indicator comprises a thiocarbamate derivative of hydroxyl coumarin.

3. The method of claim 2, wherein the thiocarbamate-based derivative is a derivative of 7-hydroxy-coumarin.

4. The method of claim 1, further comprising titrating the sample to a pH in the range of about 4.0 to about 8.5.

5. The method of claim 1, further comprising adding a phosphate buffer.

6. The method of claim 1, wherein the amount of ozone is in the range of 0-1.5 mg/L.

7. The method of claim 1, wherein the fluorescence intensity is correlated to a concentration of the ozone in the sample.

8. The method of claim 1, wherein the sample comprises water from a beverage material system.

9. The method of claim 1, further comprising addition of a co-solvent.

10. The method of claim 9, wherein the co-solvent is selected from the group consisting of: isopropanol, ethanol, poly(ethylene glycol), poly(ethylene glycol)dimethyl ether, and poly(ethylene glycol)methyl ether.

11. A method for measuring ozone in a sample, comprising:
preparing a thiocarbamate-based indicator;
introducing the thiocarbamate-based indicator to a sample, wherein the sample contains an amount of ozone and the introducing causes a change in fluorescence of the solution;
adding potassium iodide to the sample, wherein the potassium iodide accelerates the reaction rate between the thiocarbamate-based indicator and ozone and causes a change in fluorescence of the sample; and
measuring the amount of ozone in the sample by measuring the change in intensity of the fluorescence.

12. The method of claim 11, wherein the thiocarbamate-based indicator comprises a thiocarbamate derivative of hydroxyl coumarin.

13. The method of claim 12, wherein the thiocarbamate-based derivative is a derivative of 7-hydroxy-coumarin.

14. The method of claim 11, further comprising titrating the sample to a pH in the range of about 4.0 to about 8.5.

15. The method of claim 11, further comprising adding a phosphate buffer.

16. The method of claim 11, wherein the amount of ozone is in the range of 0-1.5 mg/L.

17. The method of claim 11, wherein the fluorescence intensity is correlated to a concentration of the ozone in the sample.

18. The method of claim 11, wherein the sample comprises water from a beverage material system.

19. The method of claim 11, further comprising addition of a co-solvent.

20. A measurement device which measures ozone in a sample, comprising:
a thiocarbamate-based indicator;
at least one measurement chamber;
a processor; and
a memory storing instructions executable by the processor to:
introduce the thiocarbamate-based indicator to a sample, wherein the sample contains an amount of ozone;
add potassium iodide to the sample, wherein the potassium iodide accelerates the reaction rate between the thiocarbamate-based indicator and ozone and causes a change in fluorescence of the sample; and
measure the amount of ozone in the sample by measuring the change in fluorescence of the sample.
